(19) United States
(12) Patent Application Publication
(10) Pub. No.: US 2025/0241544 A1
(43) Pub. Date: Jul. 31, 2025

(54) ELECTRICAL SENSOR AND BLOOD PRESSURE MONITORING SYSTEM

(71) Applicant: Simon Fraser University, Burnaby (CA)

(72) Inventors: Woo Soo KIM, Vancouver, BC (CA); Taeho KIM, Surrey, BC (CA)

(21) Appl. No.: 18/701,064

(22) PCT Filed: Oct. 4, 2022
(86) PCT No.: PCT/CA2022/051469

§ 371 Date: Apr. 12, 2024

(30) Foreign Application Priority Data
Oct. 14, 2021 (CA) 3,134,287

(51) Int. Cl. A61B 5/021; A61B 5/282

(52) U.S. Cl. CPC A61B 5/02125; A61B 5/02141; A61B 5/282

(57) ABSTRACT

A sensor, for measuring an electrical signal, includes a compressible body portion defining a longitudinal axis and having a foldable wall extending from a first end of the body portion to a second end of the body portion. The body portion is movable from a decompressed state to a compressed state by moving the first end along the longitudinal axis relative to the second end. The wall includes fold lines formed therein such that, during movement of the body portion from the decompressed state to the compressed state, the wall is folded along the fold lines. The sensor further includes an electrode portion connected to the body portion and comprising an electrode for measuring the electrical signal.

### FIELD

The present disclosure relates to an electrical sensor for measuring an electrical signal. The present disclosure also relates to a system that includes an electrical sensor and that may determine a blood pressure reading based on a measured electrical signal.

### BACKGROUND

Obtaining accurate blood pressure readings is an important aspect of medical diagnostics. A typical way of obtaining blood pressure readings is to use a cuff that is placed around the patient's arm and then inflated. Some patients, however, are uncomfortable with their blood pressure being measured with the use of such cuffs. Moreover, in hospital or similar settings, cuff-based blood pressure measurements generally require interaction between patients and the healthcare professional, thereby taking up valuable time from the healthcare professional.

Cuff-less blood pressure monitoring systems do exist in the form of so-called “wet electrodes” that may be placed in direct contact with the patient's skin. In order to provide improved contact between the electrode and the patient's skin, it is typical to apply an electrolytic gel between the electrode and the skin. The use of such gels, however, has its drawbacks. For example, in addition to being potentially unsanitary, the sensation of the cold gel on the patient's skin may be uncomfortable to the patient, and in some cases may irritate the patient's skin.

In view of these drawbacks associated with wet electrodes, so-called “dry electrodes” may also be used. While a dry electrode dispenses with the need for an electrolytic gel, additional elements such as an adhesive or a belt generally have to be used in order to secure the electrode to the skin and obtain good skin-to-electrode contact.

It would be beneficial if such dry electrodes could achieve good skin-to-electrode contact without the need for such additional elements.

### SUMMARY

According to a first aspect of the disclosure, there is provided a sensor for measuring an electrical signal, comprising: a compressible body portion defining a longitudinal axis and comprising a foldable wall extending from a first end of the body portion to a second end of the body portion, wherein: the body portion is movable from a decompressed state to a compressed state by moving the first end along the longitudinal axis relative to the second end; and the wall comprises fold lines formed therein such that, during movement of the body portion from the decompressed state to the compressed state, the wall is folded along the fold lines; and an electrode portion connected to the body portion and comprising an electrode for measuring the electrical signal.

The foldable wall may comprise an auxetic material.

The electrode portion may further comprise a foldable wall extending from a first end of the electrode portion to a second end of the electrode portion. The electrode portion may be movable from a unexpanded state to an expanded state by moving the first end of the electrode portion along the longitudinal axis relative to the second end of the electrode portion. The wall of the electrode portion may comprise fold lines formed therein such that, during movement of the electrode portion from the unexpanded state to the expanded state, the wall of the electrode portion is folded along the fold lines of the electrode portion and thereby causes the electrode portion to expand in a radial direction relative to the longitudinal axis.

The fold lines of the electrode portion may be arranged such that, during movement of the electrode portion from the unexpanded state to the expanded state, a distance separating the first end of the electrode portion from the second end of the electrode portion decreases in a direction defined by the longitudinal axis.

The fold lines of the electrode portion may define multiple polygonal surface portions of the wall of the electrode portion. The multiple polygonal surface portions may comprise a sequence of alternating rectangular and triangular surface portions.

The electrode portion may comprise an outer surface and an opposing inner surface facing toward the longitudinal axis, and wherein the electrode is comprised on the inner surface.

The electrode may comprise one or more serpentine conductive elements.

The one or more serpentine conductive elements may extend in a first direction, and the electrode may further comprise one or more serpentine conductive elements extending in a second direction.

The first direction may be perpendicular to the second direction.

The sensor may further comprise one or more electrical conductors connected to the electrode.

The one or more electrical conductors may pass through an interior of the body portion.

The body portion may comprise an outer surface and an opposing inner surface facing toward the longitudinal axis. The one or more electrical conductors may be in contact with the outer surface of the body portion.

The fold lines of the body portion may define at least one polygonal surface portion of the wall of the body portion.

The at least one polygonal surface portion may comprise interconnected polygonal surface portions of the wall of the body portion, and the interconnected polygonal surface portions may comprise an outer surface of the wall of the body portion.

The at least one polygonal surface portion may comprise at least one planar polygonal surface portion.

The fold lines of the body portion may define a Kresling pattern on an outer surface of the wall of the body portion.

The at least one polygonal surface portion may comprise at least one triangular surface portion.

A first angle of the at least one triangular surface portion may be from about 28° to about 32°, and a second angle of the at least one triangular surface portion may be from about 38° to about 42°.

The at least one triangular surface portion may comprise an obtuse angle.

The at least one triangular surface portion may comprise a right angle.

The fold lines of the body portion may comprise inwardly-folding fold lines and outwardly-folding fold lines defining interconnected foldable portions of the wall of the body portion. Each inwardly folding fold line may be closer to the longitudinal axis than each outwardly folding fold line.

Each foldable portion may be defined by four outwardly-folding fold lines and one inwardly folding fold line.

The four outwardly folding fold lines may define a parallelogram. The inwardly folding fold line may extend from a first corner of the parallelogram to an opposite, second corner of the parallelogram.

The inwardly folding fold line may be perpendicular to the longitudinal axis.

The fold lines of the body portion may define a Yoshimura pattern on an outer surface of the wall of the body portion.

Each foldable portion may be defined by multiple inwardly-folding fold lines meeting at a point.

Each foldable portion may be defined by six outwardly-folding fold lines. The multiple inwardly-folding fold lines may meet at the point consist of three inwardly-folding fold lines.

The fold lines of the body portion may define a Tachi-Miura pattern on an outer surface of the wall of the body portion.

The wall of the body portion may be formed by three-dimensional printing.

According to a further aspect of the disclosure, there is provided a system for measuring an electrical signal generated by a patient, comprising: any of the above-described sensors; and one or more processors communicative with the sensor and configured to receive electrical conductivity readings from the electrode of the sensor.

The one or more processors may be further configured to: measure an electrical resistance of a conductive element provided on the sensor; and based on the measured electrical resistance, determine an amount of rotation of the body portion about the longitudinal axis.

The system may further comprise a photoplethysmography (PPG) sensor, and the one or more processors may be further configured to: obtain one or more PPG signals from the PPG sensor; determine one or more electrocardiogram (ECG) signals from the electrical conductivity readings; and determine a blood pressure based on the one or more PPG signals and the one or more ECG signals.

The sensor may be comprised in a humanoid robot.

The sensor may be attached to a finger of the humanoid robot.

The sensor may be attached to a portion of the finger. The portion may define a direction of extension of the finger, and the direction of extension of the finger may be parallel to the longitudinal axis of the body portion.

The sensor may be attached to a portion of the finger. The portion may define a direction of extension of the finger, and the direction of extension of the finger may be perpendicular to the longitudinal axis of the body portion.

According to a further aspect of the disclosure, there is provided a humanoid robot comprising: a first sensor for generating an ECG reading from a patient; a second sensor for generating a PPG reading from the patient; and one or more controllers for determining, based on the ECG reading and the PPG reading, a blood pressure of the patient.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

Embodiments of the disclosure will now be described in detail in conjunction with the accompanying drawings of which:

FIGS. 1A-1C show an electrical sensor under various stages of compression, according to an embodiment of the disclosure;

FIG. 2 shows a fold line pattern of a body portion of an electrical sensor, according to an embodiment of the disclosure;

FIGS. 3A and 3B show a fold line pattern of a body portion of an electrical sensor, according to an embodiment of the disclosure;

FIG. 4A shows a body portion of an electrical sensor transitioning from a decompressed state to a compressed state, according to an embodiment of the disclosure;

FIG. 4B shows a fold line pattern of the body portion of FIG. 4A, according to an embodiment of the disclosure;

FIG. 5 shows various different body portions of electrical sensors based on different values of angles α and β, according to embodiments of the disclosure;

FIG. 6 is a plot of Poisson's ratio as a function of strain, according to an embodiment of the disclosure;

FIG. 7 is a plot of angular rotation as a function of strain, according to an embodiment of the disclosure;

FIG. 8 is a plot of stress as a function of strain for an electrical sensor according to an embodiment of the disclosure;

FIG. 9 is a plot of elastic modulus as a function of different values for the angles α and β, according to embodiments of the disclosure;

FIG. 10 shows, in various stages of compression, a body portion and an electrode portion of an electrical sensor according to an embodiment of the disclosure;

FIG. 11 is a plot of a normalized area of body and electrode portions of an electrical sensor as a function of compression, according to an embodiment of the disclosure;

FIG. 12 is a plot of angular rotation of a body portion of an electrical sensor as a function of compression of the body portion, according to an embodiment of the disclosure;

FIG. 13 is a plot of strain as a function of compression of an electrode portion of an electrical sensor, according to an embodiment of the disclosure;

FIG. 14 is a plot of impedance as a function of strain, according to an embodiment of the disclosure;

FIG. 15 is a schematic diagram of an electrode formed on an electrode portion of an electrical sensor, according to an embodiment of the disclosure;

FIG. 16 shows images of serpentine, conductive elements formed on a surface of an electrode portion of an electrical sensor, according to an embodiment of the disclosure;

FIG. 17 shows a body portion of an electrical sensor in various stages of compression, according to an embodiment of the disclosure;

FIG. 18 shows electrical paths provided on a body portion of an electrical sensor, according to an embodiment of a disclosure;

FIG. 19 is a plot of electrical resistance as a function of strain applied to an electrical sensor, according to an embodiment of the disclosure;

FIG. 20 is a plot of electrical resistance as a function of time, before and after a gripping operation, according to an embodiment of the disclosure;

FIG. 21 is a plot of angular rotation as a function of strain applied to an electrical sensor, for different values for the angles α and β, according to an embodiment of the disclosure;

FIG. 22 is a schematic diagram of a blood pressure monitoring system, according to an embodiment of the disclosure;

FIG. 23 shows a humanoid robot comprising a blood pressure monitoring system, according to an embodiment of the disclosure;

FIG. 24 shows an electrical sensor connected at different angles to a finger of a humanoid robot, according to an embodiment of the disclosure;

FIG. 25 shows plots of voltage as a function of time for an electrical sensor according to an embodiment of the disclosure and for a traditional Ag/AgCl electrode;

FIG. 26 shows plots of voltage as a function of time, indicative of heart rate as measured using an electrical sensor according to an embodiment of the disclosure;

FIG. 27 is a plot of photoplethysmogram (PPG) and electrocardiogram (ECG) signals as a function of time, according to an embodiment of the disclosure;

FIG. 28 is a plot of systolic blood pressure (SBP) as a function of pulse arrival time (PAT), according to an embodiment of the disclosure; and

FIG. 29 is a plot of measured and estimated SBP and diastolic blood pressure (DBP) for different postures and exercises, according to an embodiment of the disclosure.

### DETAILED DESCRIPTION

The present disclosure seeks to provide an improved electrical sensor and system for monitoring a blood pressure of a patient. While various embodiments of the disclosure are described below, the disclosure is not limited to these embodiments, and variations of these embodiments may well fall within the scope of the disclosure which is to be limited only by the appended claims.

Generally, embodiments of the disclosure are directed at an electrical sensor that may be used to measure one or more electrical signals generated by a patient. According to some embodiments, the sensor comprises a cylindrical body portion with a foldable outer wall comprising an arrangement of fold lines defining a fold line pattern. The fold line pattern may comprise any one or more of multiple different types of origami-based or non-origami-based patterns, such as a Kresling pattern, a Yoshimura pattern, or a Tachi-Miura pattern. The sensor may be printed using any suitable three-dimensional printing technology, and is therefore easily replaceable.

The sensor further includes an electrode portion connected to the body portion. An electrode is provided on the electrode portion, for example on an interior surface of the electrode portion. The electrode may comprise one or more serpentine-shaped conductive elements. In order to achieve good skin-to-electrode contact between the sensor and the patient, the electrode portion may also comprise an outer wall with a pattern of fold lines designed such that, during compression of the body portion, the electrode portion expands outwardly and may expose the electrode for contact with the patient's skin. Compression of the body portion may further result in the generation of a vacuum localized within the interior of the sensor. The vacuum may assist the electrode portion in attaching to the patient, for example in a similar way that a leech may attach itself to the skin of a host. In order to assist with the generation of the vacuum and thereby provide a strong seal to the patient's skin, the body portion of the sensor may comprise one or more auxetic materials (for example, materials having a tubular shape and a negative Poisson's ratio). Examples of auxetic materials include, as noted above, Kresling, Yoshimura, and Tachi-Miura structures, as well as re-entrant structures.

The sensor may be cost-effectively produced through three-dimensional (3D) printing. Because of the cost-effective way in which the sensor may be produced, sensors having different parameters (such as different volumes, or different elastic moduli) may be rapidly and easily produced.

The sensor may be deployed as part of a system that is configured to measure the patient's blood pressure using an electrocardiogram (ECG) signal based on the measured one or more electrical signals. For example, according to some embodiments, the sensor may be incorporated in one or more fingers of a humanoid robot. The humanoid robot may include a photoplethysmogram (PPG) sensor that may be used to obtain a PPG signal from the patient. The PPG sensor measures changes in oxygen concentration in the patient's blood at a distal location on the patient (for example, at a finger of the patient). Based on the ECG and the PPG signals obtained by the respective sensors, a processor may determine the patient's blood pressure. For example, the patient's blood pressure may be determined using one or more of the methods described in “The use of photoplethysmography for assessing hypertension”, Elgendi, M. et al., NPJ. Digit. Med. 2, 60 (2019), incorporated herein by reference in its entirety.

The robot may resemble a human in order to create a friendly environment for those patients who may feel isolated or may be suffering from conditions such as delirium. The use of a humanoid robot may put such patients at ease, and provide a more comfortable environment in which vital signs (such as blood pressure) of the patient may be monitored without the need for cuff-based blood pressure monitoring devices.

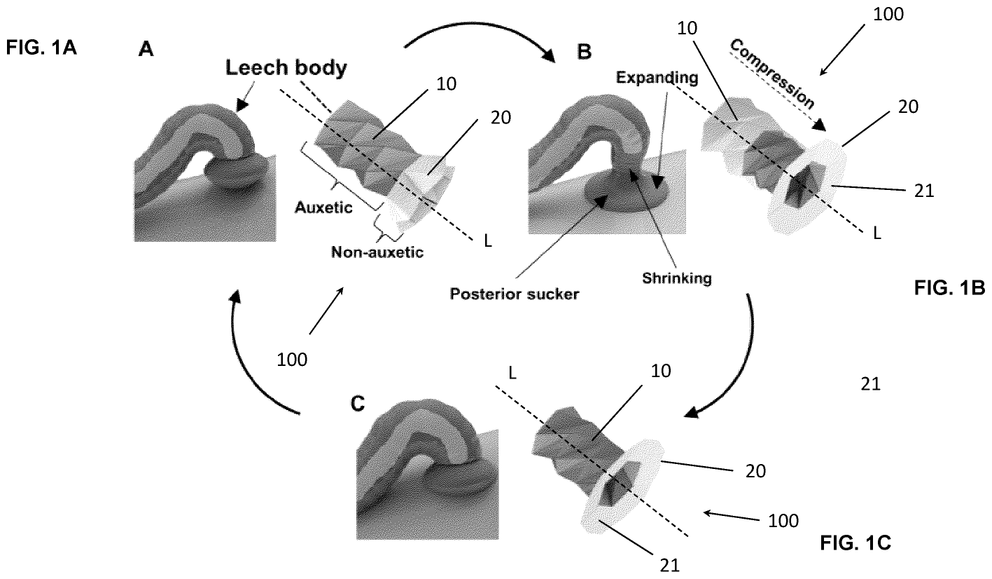

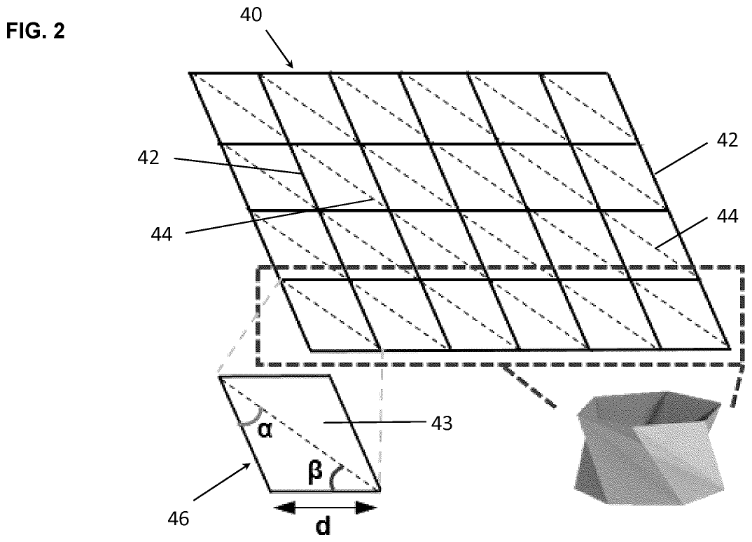

Turning to FIGS. 1A-1C, there is shown in various states of compression a sensor 100 according to an embodiment of the disclosure. Sensor 100 includes a cylindrical body portion 10 and an electrode portion 20 connected to an end of body portion 10. As described in further detail below, body portion 10 comprises a foldable outer wall with a pattern of fold lines formed in the outer wall. During compression of body portion 10 by a force acting in a direction of a longitudinal axis L defined by body portion 10, the outer wall is configured to fold along the fold lines, and a height of body portion 10 decreases. The outer wall may be made of one or more auxetic materials, such as a Kresling, Yoshimura, or Tachi-Miura structure formed by a series of tessellated triangles. Although not visible in FIG. 1, one or more electrodes described in further detail below are provided on an inner surface 21 of electrode portion 20 (that is, a surface facing inwardly toward longitudinal axis L).

Although not shown in FIG. 1, one or more electrical conductors may connect the electrode provided on electrode portion 20 to one or more processors that may obtain and process electrical conductivity readings obtained from the electrode. The one or more electrical conductors may pass through an interior of sensor 100 (that is, within the internal volume defined by the outer wall of body portion 10) and/or may pass along an exterior surface of body portion 10.

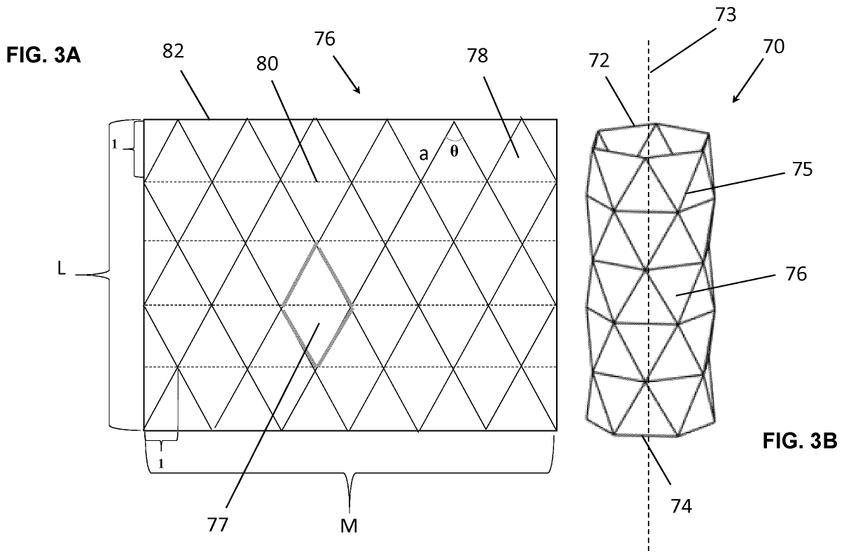

The pattern of fold lines provided on the outer wall of body portion 10 may be made according to one or more suitable origami designs. For example, turning to FIG. 2, a pattern of fold lines on the outer surface of a wall 40 of body portion 10 may comprise a Kresling pattern, shown in more detail in FIG. 2. FIG. 2 shows in particular the three-dimensional wall 40 of body portion 10, shown in FIGS. 1A-1C, transposed to a two-dimensional surface. Two-dimensional wall 40 is shaped as a parallelogram comprising a grid of horizontally and generally vertically aligned folding portions 46. Each folding portion 46 is parallelogram-shaped and is defined by four outwardly-folding fold lines 42 and one inwardly-folding fold line 44 extending from a first corner of the parallelogram defined by folding portion 46 to a second, opposite corner of the parallelogram defined by folding portion 46. Generally, an outwardly-folding fold line 42 may be defined as a fold line that folds outwardly during compression of body portion 10, whereas an inwardly-folding fold line 44 may be defined as a fold line that folds inwardly during compression of body portion 10. Outwardly folding fold lines 42 are located further from longitudinal axis 15 than inwardly folding fold lines 44.

Each foldable portion 46 defined on two-dimensional wall 40 comprises a pair of planar, polygonal (in this case, triangular) surface portions 43 defined by the intersection of the inwardly folding fold line 44 with the four outwardly folding fold lines 42. The totality of interconnected, triangular surface portions 43 form the outer surface of wall 40. Each triangular surface portion 43 is defined by two angles, α and β. As described in further detail below, α and β may be adjusted to alter one or more parameters of body portion 10.

According to the embodiment of FIG. 2, a height of two-dimensional wall 40 is 7 mm and a width of two-dimensional wall 40 is 5 mm, with α=30° and β=40°. According to other embodiments of the disclosure, the height and width of the wall forming body portion 10 may be adjusted to alter the volume of air contained within sensor 100.

In addition to the Kresling pattern shown in FIG. 2, a body portion 10 may be formed according to other patterns of fold lines. For example, turning to FIGS. 3A and 3B, there is shown a body portion 70 defining a longitudinal axis 73 and having a foldable wall 76 extending between a first end 72 and a second end 74, according to another embodiment of the disclosure. According to this embodiment, the pattern of fold lines 75 on the outer surface of wall 76 may comprise a Yoshimura pattern, shown in more detail in FIG. 3A. FIG. 3A shows in particular the three-dimensional wall 76 of FIG. 3B transposed to a two-dimensional surface. Two-dimensional wall 76 is shaped as a rectangle comprising an arrangement of folding portions 77. Each folding portion 77 is parallelogram-shaped and is defined by four outwardly-folding fold lines 82 and one inwardly-folding fold line 80 extending from a first corner of the parallelogram defined by folding portion 77 to a second, opposite corner of the parallelogram defined by folding portion 77. Furthermore, inwardly-folding fold line 80 extends perpendicularly to longitudinal axis 73 defined by body portion 70.

Each foldable portion 77 defined on two-dimensional wall 76 comprises a pair of planar, polygonal (in this case, triangular) surface portions 78 defined by the intersection of the inwardly folding fold line 80 with the four outwardly folding fold lines 82. The totality of interconnected, triangular surface portions 78 form the outer surface of wall 76. Each triangular surface portion 78 is defined by an angle, θ=60°. During compression of body portion 70, first end 72 is not rotated relative to second end 74 about longitudinal axis 73.

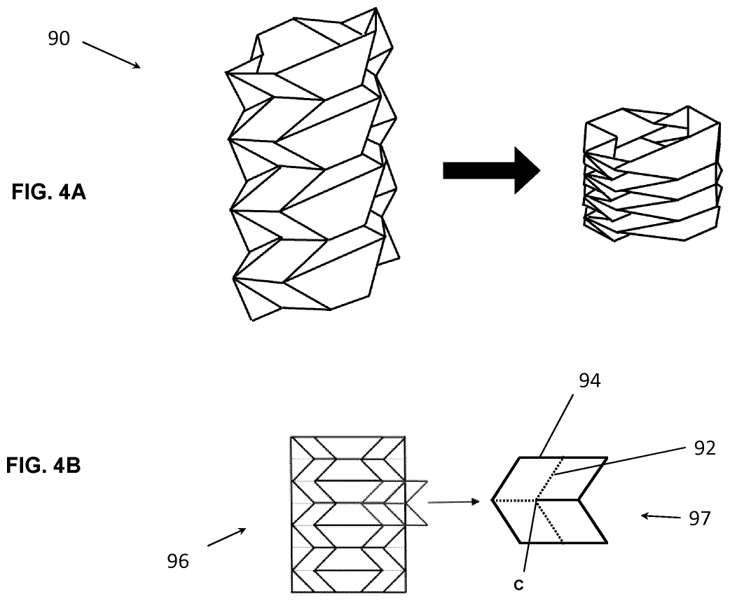

In addition to the Yoshimura pattern shown in FIG. 3A, a sensor may be formed according to still other patterns of foldable portions. For example, turning to FIGS. 4A and 4B, there is shown a body portion 90 for an electrical sensor, according to an embodiment of the disclosure. Body portion 90, shown in FIG. 4A, has a corresponding foldable wall 96 shown in FIG. 4B. According to this embodiment, the pattern of fold lines on the outer surface of wall 96 comprises a Tachi-Miura pattern. FIG. 4B shows in particular the three-dimensional wall 96 of FIG. 4A transposed to a two-dimensional surface. Two-dimensional wall 96 is shaped as a rectangle comprising an arrangement of folding portions 97. Each folding portion 97 is defined by six outwardly-folding fold lines 94 and three inwardly-folding fold lines 92. The three inwardly-folding fold lines 92 meet at a point O1 as can be seen in FIG. 4B. During compression of body portion 90, body portion 90 does not rotate about the longitudinal axis defined by airbag 90.

It will be recognized by the skilled person that any number of suitable fold line patterns may be used in order to form a body portion according to the present disclosure.

As described above, body portion 10 is connected to electrode portion 20 at a distal end of sensor 100. Electrode portion 20 also includes a pattern of fold lines formed within an outer wall of electrode portion 20. The pattern of fold lines is configured such that, when electrode portion 20 is applied against the skin of a patient, and during compression of body portion 10 along longitudinal axis L, electrode portion 20 expands radially outwardly, away from longitudinal axis L, so as to expose a greater proportion of inner surface 21 to the patient's skin. This may enable a greater proportion of the electrode provided on inner surface 21 to come into contact with the patient's skin.

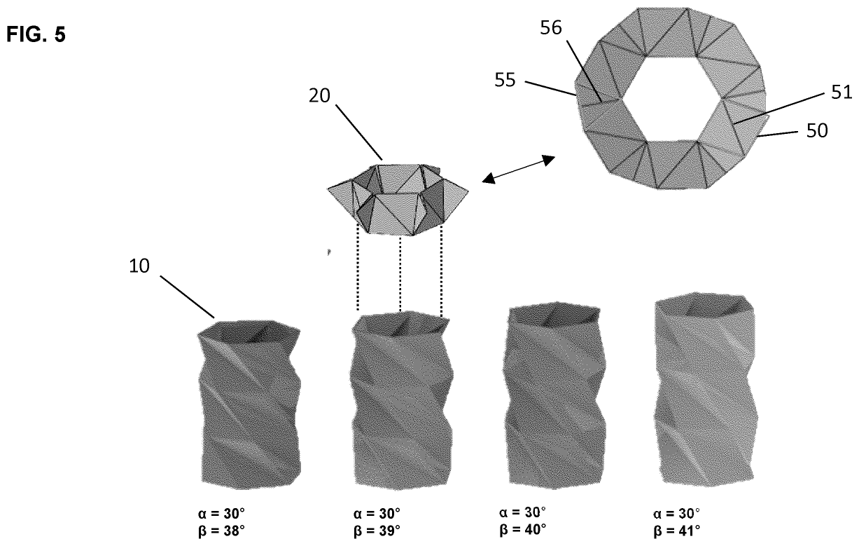

Turning to FIG. 5, there are shown different body portions 10 of an electrical sensor according to different values selected for the angles α and β. In addition, electrode portion 20 is shown in its fully radially expanded state, illustrating in greater detail the pattern of fold lines formed within electrode portion 20. In particular, the outer wall of electrode portion 20 includes a number of rectangular surface portions 50 alternating with a number of interconnected triangular surface portions 55. Each rectangular surface portion 50 is bisected by a fold line 51 dividing the rectangular surface portion 50 into a pair of right triangles, and each triangular surface portion 55 is bisected by a fold line 56 dividing the triangular surface portion into another pair of right triangles.

It will be recognized by the skilled person that any number of suitable fold line patterns may be used in order to form an electrode portion according to the present disclosure.

As described above, body portion 10 may be formed of one or more auxetic materials. On the other hand, according to some embodiments, electrode portion 20 is formed of one or more materials that are not auxetic, although electrode portion 20 may also be formed of one or more auxetic materials if desired.

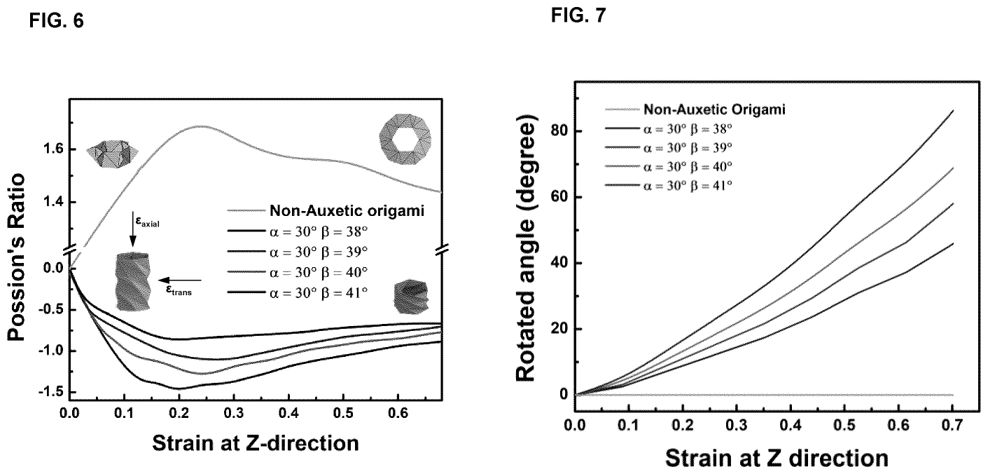

FIG. 6 shows a plot of Poisson's ratio for body portion 10 and electrode portion 20 for different values of α and β, as a function of strain applied to body portion 10 and electrode portion 20 in a direction of longitudinal axis L.

FIG. 7 shows a plot of angular rotation of body portion 10 relative to longitudinal axis L during compression of body portion 10, for different values of α and β. As can be seen, electrode portion 20 (“non-auxetic origami”) does not undergo rotation.

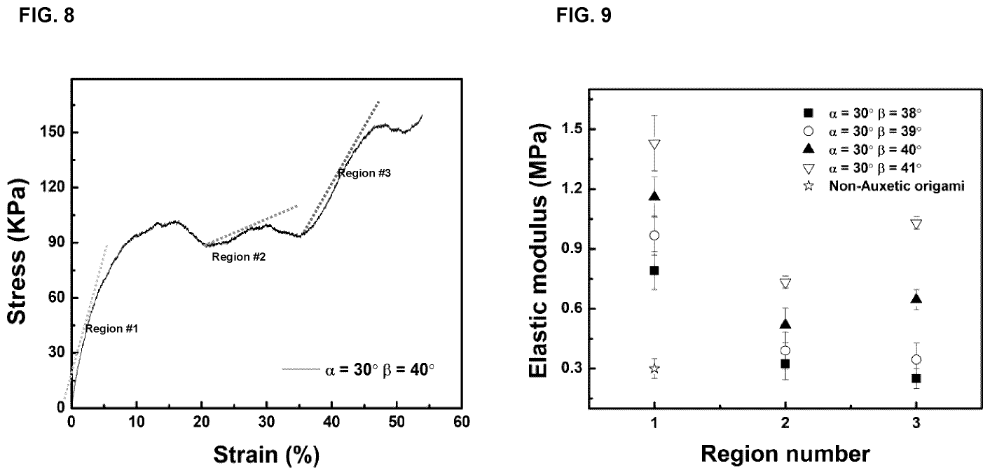

FIG. 8 shows the stress-stain curve for body portion 10 with the values α=30° and β=40°. During the compression, the stress increases cyclically (region #1 to region #3) depending on the applied strain. Each sudden increase in stress generally correlates to a different horizontal row of folding portions 46 undergoing folding. Thus, the body portion 10 exhibits three different stable states at roughly 15%, 30%, and 45% of applied strain. Body portion 10 is generally stressed at its fold lines 41 rather than along polygonal surface portions 43. As a result, when body portion 10 is exposed to cyclic, compressive loading, fatigue accumulated in body portion 10 may be relatively smaller and the mechanical reliability of body portion 10 may be improved since the applied stress is concentrated at fold lines 41 of each row of folding portions 46, with folding portions 46 being generally better configured to absorb the applied stress.

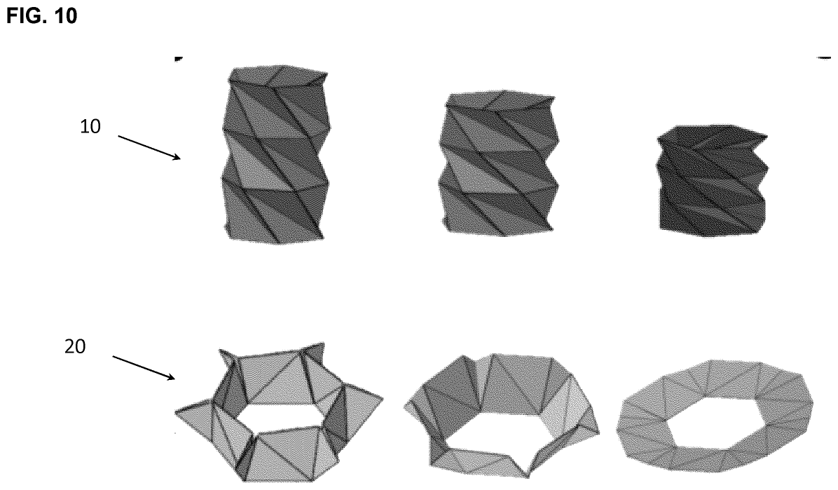

FIG. 9 is a plot of the elastic modulus of body portion 10 and electrode portion 20 for different values of α and β. Generally, the greater the value of the angle β, the greater the elastic modulus. Body portion 10 with β=38° has 0.79±0.090, 0.33±0.081, and 0.25±0.054 MPa for regions #1-#3, respectively. Body portion 10 with β=39° has 0.98±0.096, 0.39±0.082, and 0.35±0.078 MPa for regions #1-#3, respectively.

As can be seen from FIGS. 6-9, it was found that the sensor with the highest Poisson ratio had α=30° and β=41°. However, for such settings of the angles α and β, the elastic modulus was determined to be too high, and therefore it may be preferable for the body portion to have α=30° and β=40°.

Turning to FIG. 10, there are shown schematics of the mechanical behaviors of body portion 10 and electrode portion 20 of sensor 100, during compression.

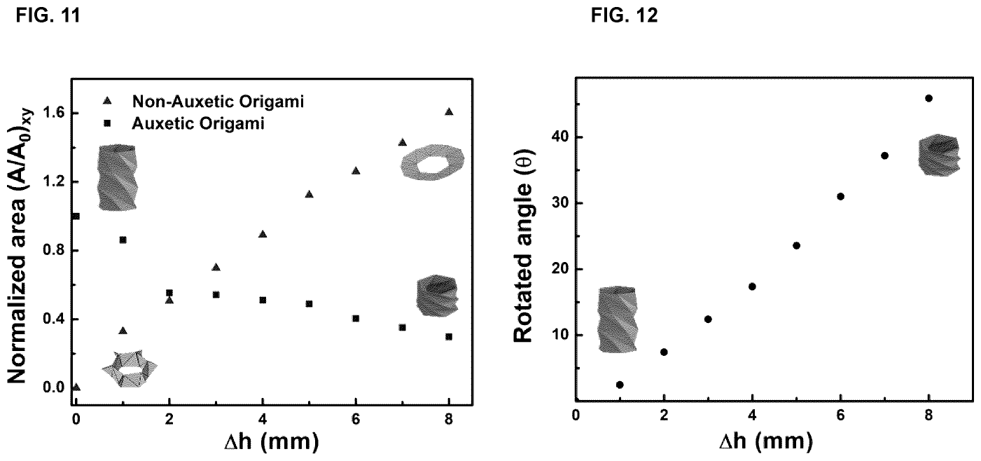

FIG. 11 shows a plot of a normalized area of body portion 10 and electrode portion 20, as a function of compression (i.e. the change in height h of electrical sensor 100). The normalized area may be calculated based on the area of a plane within electrical sensor 100 bisecting perpendicularly the longitudinal axis L, when electrical sensor 100 is undergoing compression, divided by the area of the plane when electrical sensor 100 is not compressed.

FIG. 12 shows a plot of angular rotation of body portion 10 about longitudinal axis L of electrical sensor 100, as a function of compression of body portion 10.

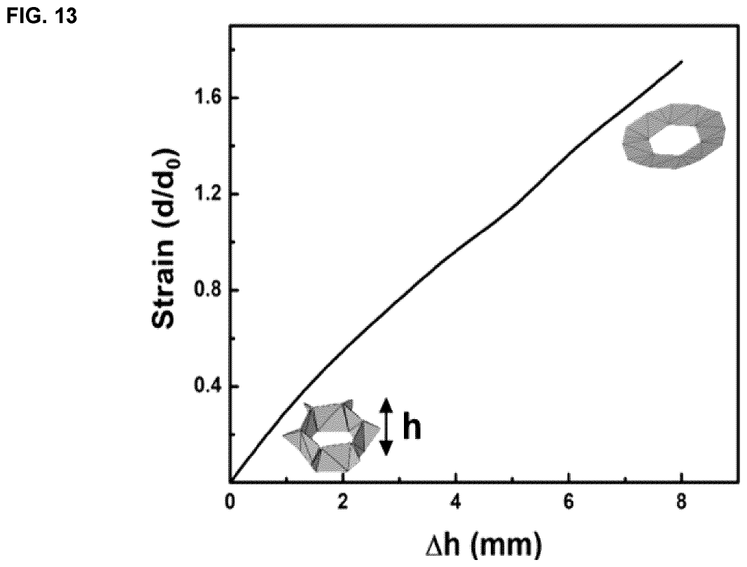

FIG. 13 shows a plot of strain experienced by electrode portion 20 as a function of compression of electrode portion 20 about longitudinal axis L.

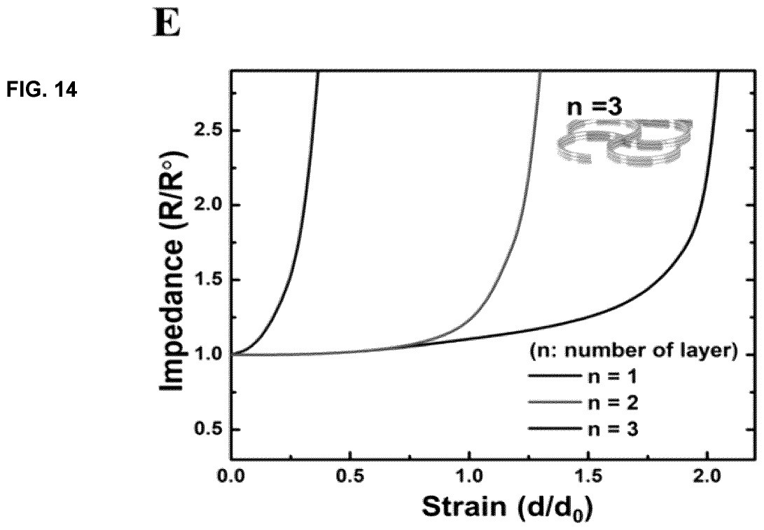

FIG. 14 shows a plot of the electrical impedance of the electrode as a function of strain experienced by electrode portion 20. The number of layers refers to the number of stacked layers of serpentine conductive elements forming the electrode, as will be described in greater detail below.

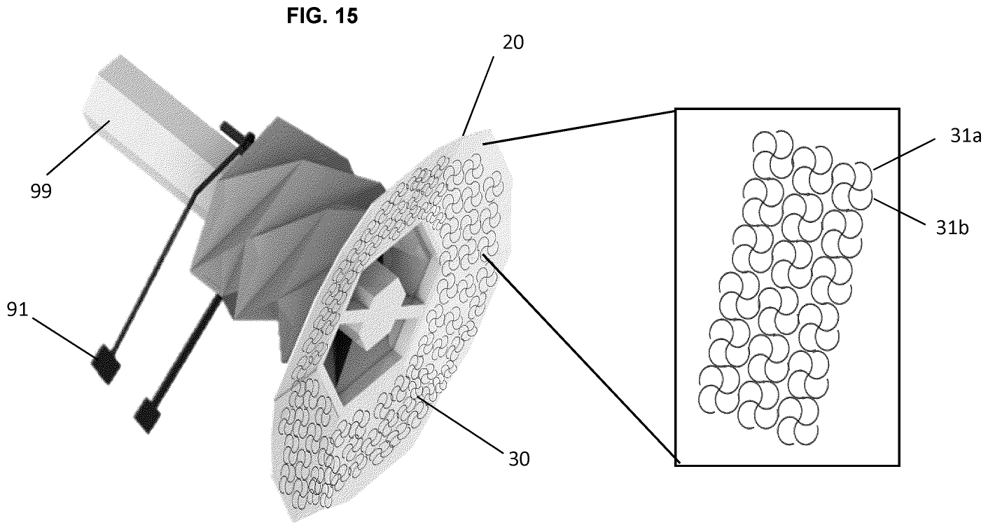

Turning to FIG. 15, there is shown the underside of electrode portion 20 with an electrode 30 printed thereon. Electrode portion 20 is shown in a generally fully radially expanded configuration. Electrode 30 comprises a grid of serpentine conductive elements. In particular, the grid consists of a number of first conductive elements 31a extending in a first direction, and a number of second conductive elements 31b extending in a second direction perpendicular to the first direction. This configuration may maximize the surface area of electrode 30 that contacts the patient's skin. According to other embodiments, any of various other suitable arrangement of conductive elements may be used for the electrode. For example, according to some embodiments, the first conductive elements may extend in a first direction that is angled relative to, but not perpendicular to, a second direction in which extend the second conductive elements.

FIG. 15 further shows a guide rod 99 for guiding motion of body portion 10 and electrode portion 20 during compression of body portion 10, and a conductive path 91 described in further detail below.

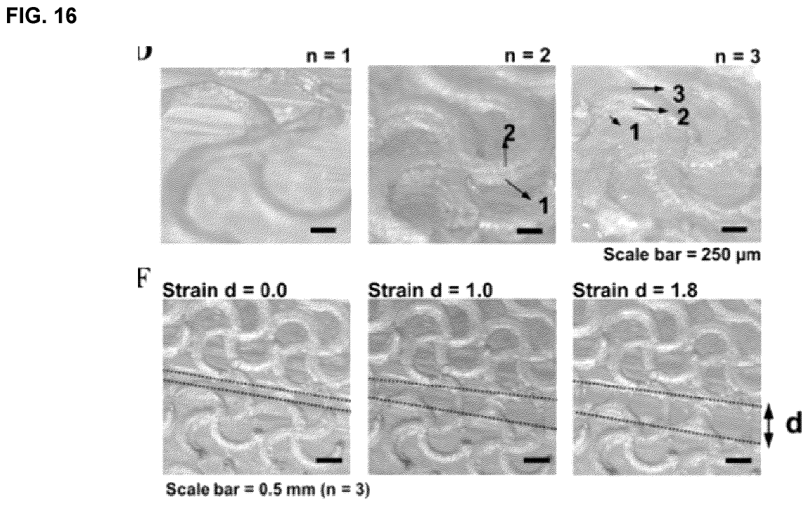

FIG. 16 shows images, taken at various degrees of magnification, of the serpentine, conductive elements printed on the inner surface of the electrode portion of a sensor. The integer “n” indicates the number of stacked layers of serpentine conductive elements forming the electrode. The arrows refer to the layer number of the conductive elements. According to some embodiments, all layers are printed using the same material.

When a Kresling fold pattern is employed for the body portion of the sensor, the ends of the body portion rotate relative to one another during compression of the body portion. Accordingly, based on the degree of rotation that the body portion experiences, it is possible to estimate the degree of compression that the body portion undergoes, which in turn may allow one determine whether a sufficient vacuum has been generated so as to enable the sensor to adhere to the patient's skin.

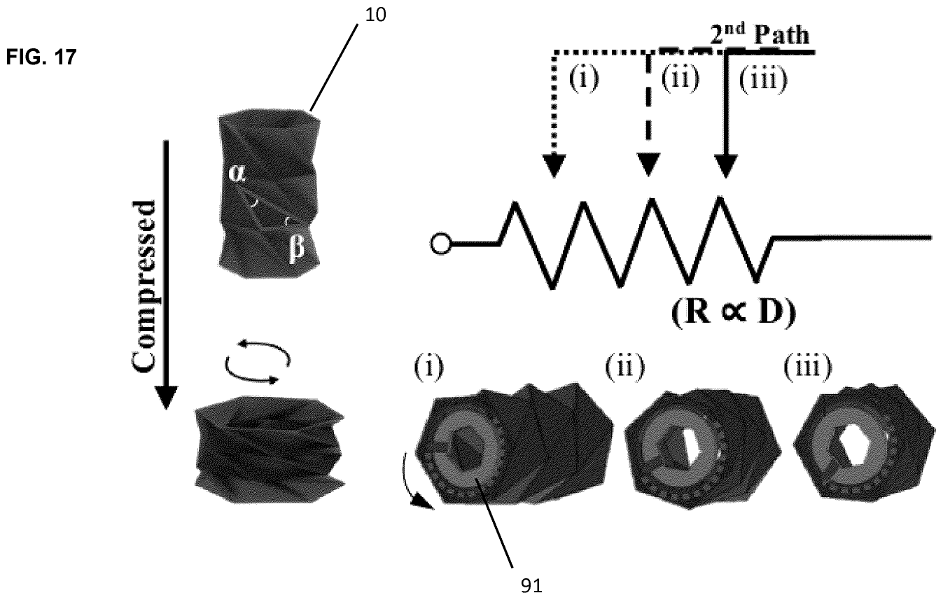

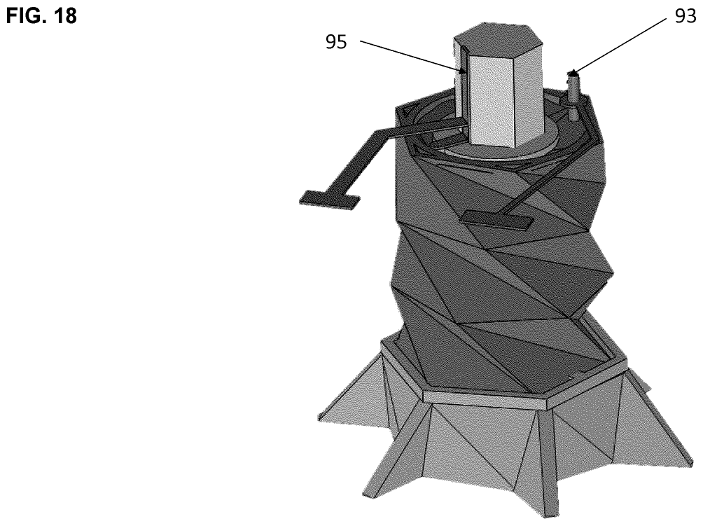

Accordingly, with reference to FIGS. 17 and 18, a conductive path 91 may be provided on body portion 10. A pair of conductive pins 93 and 95 or other conductive elements (FIG. 18) may be provided at either end of conductive path 91. Pin 93 is fixed relative to the outer wall of body portion 10 and therefore rotates with body portion 10 during compression of body portion 10. Pin 95 is not fixed relative to the outer wall of body portion 10 and therefore does not rotate with body portion 10 during compression of body portion 10. As body portion 10 is compressed, the rotating behavior of body portion 10 changes the relative position between the pair of conductive pins 93 and 95.

Therefore, by monitoring the electrical resistance of conductive path 91 extending between pin 93 and pin 95, it is possible to determine the degree of compression of body portion 10 as a function of the change in electrical resistance that is observed. Accordingly, based on the change in electrical resistance that may be observed, the degree of rotation of body portion 10 may be determined, and one may determine whether sufficient suction has been generated at the end of the sensor such that good skin-electrode contact has been made.

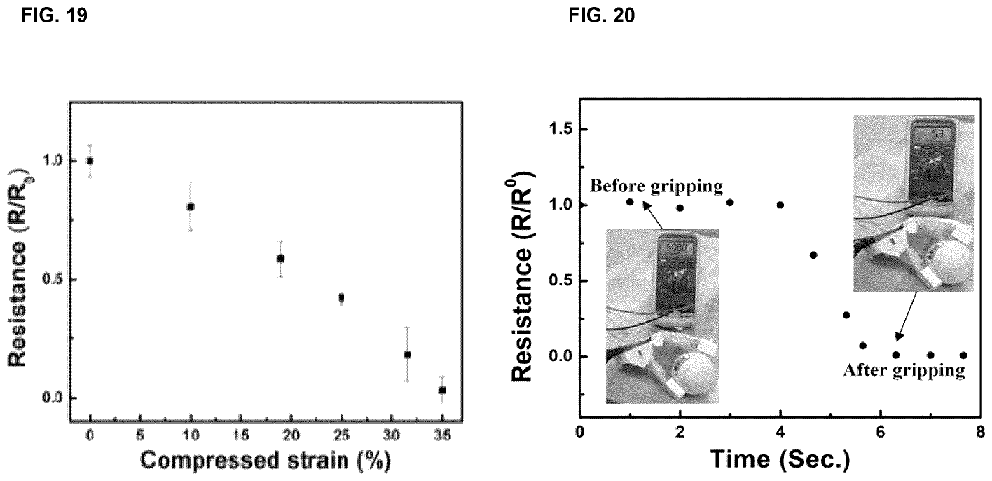

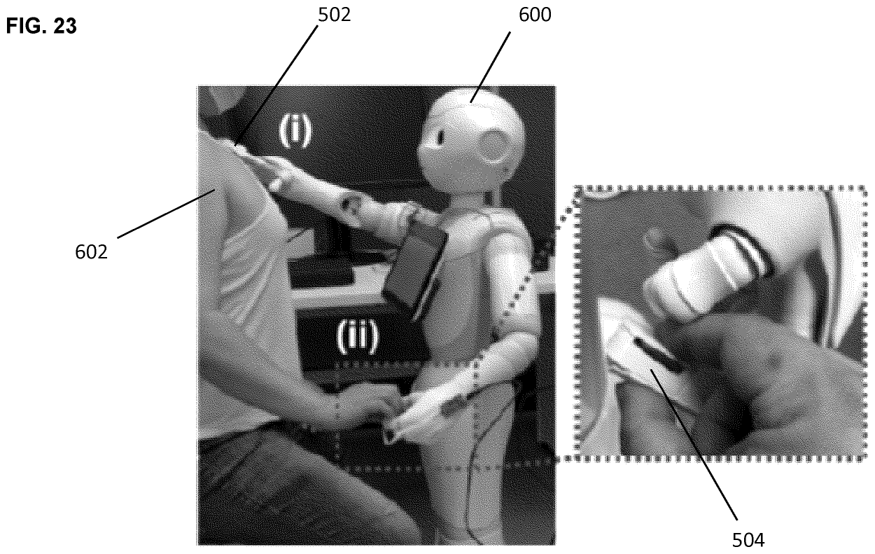

In this context, FIG. 19 shows a plot of measured electrical resistance decreasing as a function of strain applied to the sensor in the direction of the longitudinal axis of the sensor. Similarly, FIG. 20 shows the change in electrical resistance that is observed when a robotic hand grips an object (a tennis ball). In particular, one or more of the electrical sensors are provided in the fingers of the robotic hand (for example as can be seen in FIG. 23). When the robotic hand grips the ball, the sensors are compressed and the electrical resistance changes, thereby providing an indication of the degree of grip.

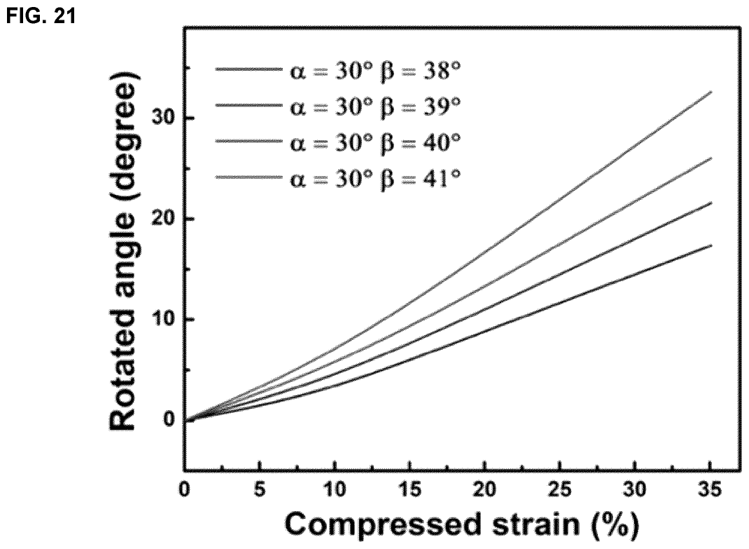

The angles α and β may be selected so as to linearly optimize the rotation of body portion 10 as a function of longitudinal compression of the sensor. For example, as can be seen in FIG. 21, according to some embodiments, the least degree of non-linearity was found with β=40°. For the angle α, the minimum angle required to generate a working origami structure was found to be 30°. Therefore, according to some embodiments, the optimum angles for α and β may be 30° and 40°, respectively.

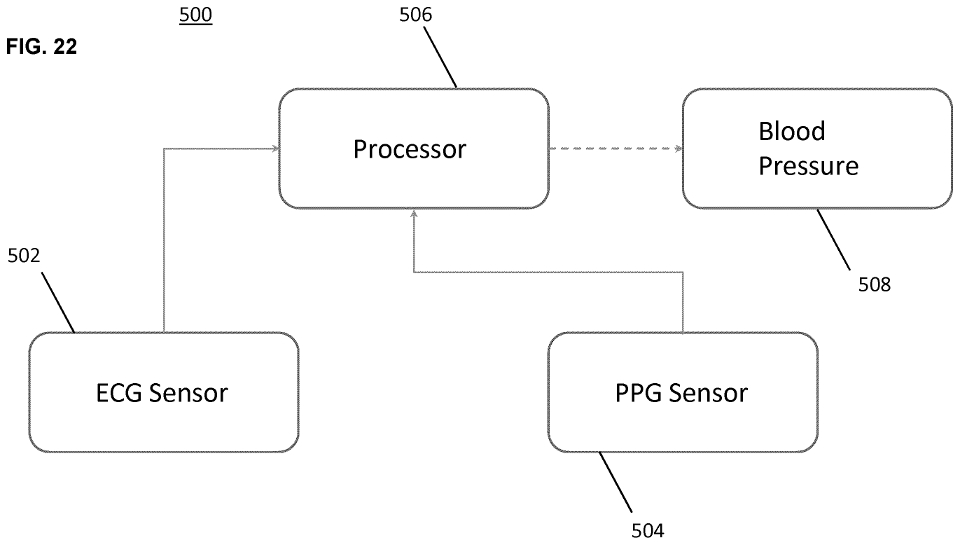

As will now be described in further detail, an electrical sensor as described above may be included in a blood pressure monitoring system for measuring a blood pressure of a patient. FIG. 22 shows a schematic diagram representing an embodiment of such a system. In particular, a blood pressure monitoring system 500 includes an ECG sensor 502 (such as sensor 100 described above), a PPG sensor 504, and a processor 506 communicatively coupled to ECG sensor 502 and PPG sensor 504. Processor 506 may obtain ECG and PPG signals from ECG sensor 502 and PPG sensor 504 that are attached to a patient. Processor 506 may then process the signals to determine a blood pressure 508 of the patient.

Turning to FIG. 23, there is shown an example of blood pressure monitoring system 500 incorporated in a humanoid robot 600. Humanoid robot 600 is used to determine a blood pressure of a patient 602, in accordance with an embodiment of the disclosure. As described above in the more general context of FIG. 22, robot 600 includes one or more ECG sensors 502 (such as sensor 100) provided on one or more fingers of robot 600. In addition, robot 600 includes one or more PPG sensors 504 provided, for example, on one or more other fingers of robot 600. PPG sensors 504 may be any of various sensors known to those of skill in the art, such as a pulse oximeter. As can be seen from FIG. 23, ECG sensor 502 is being used to measure electrical signals generated by the patient's heart, while PPG sensor 504 is being used to measure an oxygen saturation within blood of patient 602.

Robot 600 may include a processor (not shown) communicatively coupled to ECG sensor 502 and PPG sensor 504 to receive the readings obtained by ECG sensor 502 and PPG sensor 504. The processor may determine a blood pressure of patient 602 based on the ECG and PPG signals. Generally, the determination of blood pressure may be based on the time taken for blood to travel from the heart to the location at which PPG sensor 504 is located. This “time delay” may be referred to as a pulse arrival time (PAT) and may be calculated based on the time difference between an R-peak in the ECG signal (e.g. the most distinctive peak in the ECG signal) and a characteristic point in the PPG signal. According to some embodiments, the processor may be provided remote to robot 600, and the ECG and PPG readings may be communicated to the external processor using, for example, wired or wireless means.

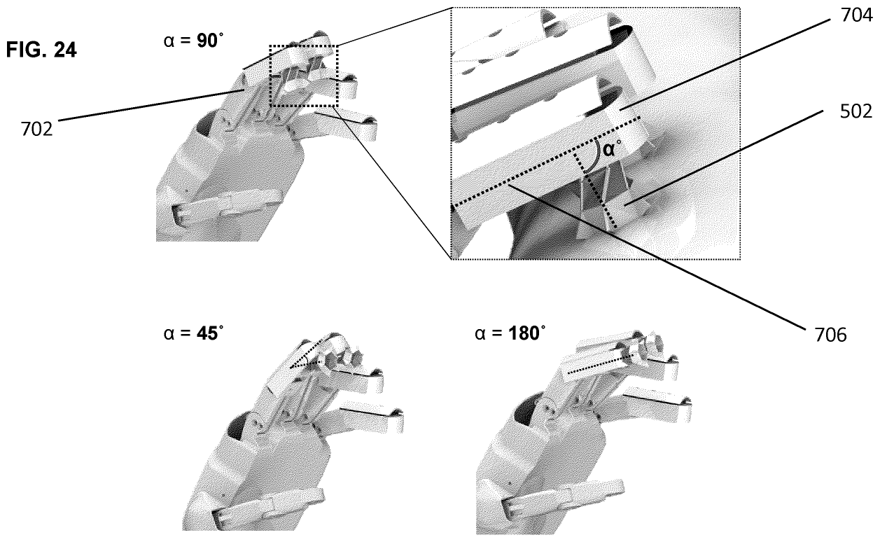

FIG. 24 shows examples of how ECG sensors 502 may be incorporated in the fingers 702 of a humanoid robot. As can be seen, each finger 702 includes a finger portion 704 defining a direction of extension 706 of finger 702. ECG sensors 502 may be provided in-line with the direction of extension 706 of finger 702 such that the direction of extension 706 is parallel to the longitudinal axes of ECG sensors 502. Alternatively, ECG sensors 502 may be provided perpendicular to the direction of extension 706 of finger 702 such that the direction of extension 706 is perpendicular to the longitudinal axis of ECG sensors 502. Alternatively still, ECG sensors 502 may be provided at some other angle (such as 45 degrees) to the direction of extension 706 of finger 702.

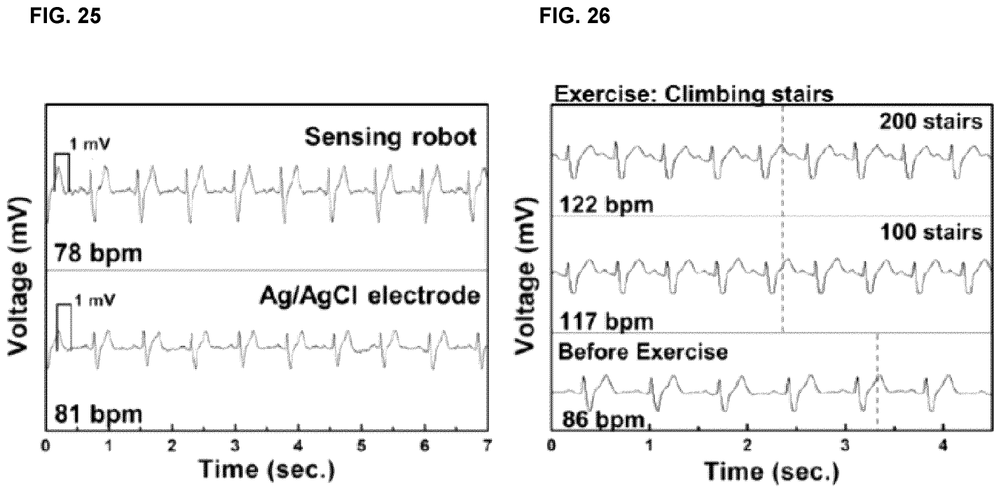

FIG. 25 shows a comparison of heart rate as measured using an electrical sensor in accordance with an embodiment of the disclosure and as measured using a traditional AG/AgCl electrode. The ECG sensing performance of an electrical sensor as described herein is comparable to results from conventional Ag/AgCl electrodes. FIG. 26 shows heart rate as measured both during and after exercise, using an electrical sensor as described herein.

FIG. 27 shows plots of ECG and PPG signals and the PAT obtained therefrom.

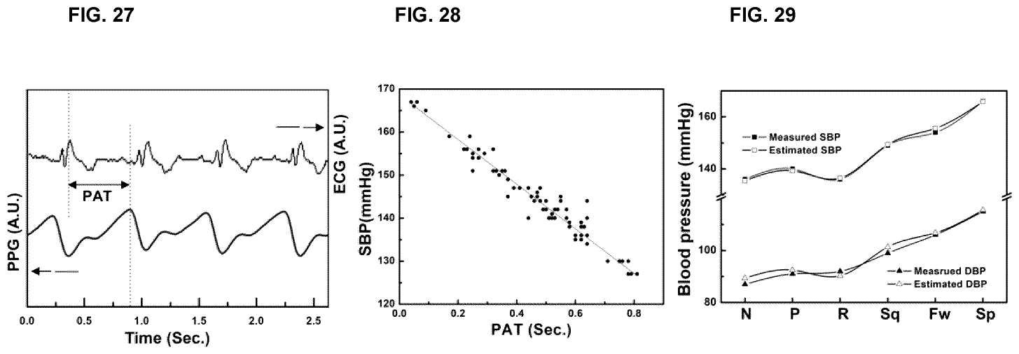

FIG. 28 shows monitored systolic blood pressure (SBP) obtained from a sphygmomanometer, as a function of the PAT. A linear fit model was used to obtain the estimated SBP, and diastolic blood pressure (DBP) is estimated as well.

FIG. 29 shows a comparison of measured and estimated SBP and DBP for different postures and under difference exercise conditions. N: Normal, P: Push-up, R: Rest, Sq: Squat, Fw: Fast Walking, and Sp: Sprint.

In order for the electrical sensor to be 3D-printed, a variety of different materials may be used. For example, according to embodiments of the disclosure, any one or more of the following various materials may be used: a thermoplastic styrenic block copolymer-based filament; a thermoplastic olefinic elastomer-based filament; a thermoplastic vulcanizate-based filament; a thermoplastic elastomer-based filament; a flexible thermoplastic copolyester-based filament; a thermoplastic polyamide-based filament; a plasticized copolyamide thermoplastic elastomer filament; and a thermoplastic polyurethane-based filament.

In order to print an electrical sensor (including a body portion and an electrode portion) as described herein, designs of the foldable walls to be used for the body portion and the electrode portion may be programmed using, for example, a suitable computer programming tool. The design may be stored on a computer-readable medium and, when read by a 3D printing machine, may enable the 3D printing machine to print the sensor according to the stored design.

While the sensor described herein has been described in the context of a body portion connected to an electrode portion, it shall be understood that, according to some embodiments, the electrode portion may consist of the electrode, in which case the electrode may be applied directly to the body portion. In other words, the disclosure extends to embodiments in which the body portion comprises the electrode, in which case the electrode itself wholly constitutes the electrode portion.

Furthermore, according to some embodiments, the electrical sensor described herein may be used in combination with an electroencephalogram (EEG) monitoring system, for monitoring brain activity of a patient.

As described above, the use of a humanoid robot for measuring blood pressure may present certain advantages over cuff-based blood pressure measuring devices. For example, due to a globally ageing population and the prevalence of heart-related disease, the demand for at-home health aides is increasing. Therefore, sensing robot applications with cuff-less blood pressure monitoring may be advantageous for the remote monitoring of blood pressure. Furthermore, blood pressure monitoring is generally required to diagnose human cardiac conditions under various environmental conditions. By using electrical sensors as described herein and that may provide improved conformal contact between the sensor and the patient, improved monitoring of patient blood pressure may be enabled.

The word “a” or “an” when used in conjunction with the term “comprising” or “including” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

The terms “coupled”, “coupling” or “connected” as used herein can have several different meanings depending on the context in which these terms are used. For example, as used herein, the terms coupled, coupling, or connected can indicate that two elements or devices are directly connected to one another or connected to one another through one or more intermediate elements or devices via a mechanical element depending on the particular context. The term “and/or” herein when used in association with a list of items means any one or more of the items comprising that list.

As used herein, a reference to “about” or “approximately” a number or to being “substantially” equal to a number means being within +/−10% of that number.

While the disclosure has been described in connection with specific embodiments, it is to be understood that the disclosure is not limited to these embodiments, and that alterations, modifications, and variations of these embodiments may be carried out by the skilled person without departing from the scope of the disclosure.

It is furthermore contemplated that any part of any aspect or embodiment discussed in this specification can be implemented or combined with any part of any other aspect or embodiment discussed in this specification.

1. A sensor for measuring an electrical signal, comprising:
a compressible body portion defining a longitudinal axis and comprising a foldable wall extending from a first end of the body portion to a second end of the body portion,
wherein:
the body portion is movable from a decompressed state to a compressed state by moving the first end along the longitudinal axis relative to the second end; and
the wall comprises fold lines formed therein such that, during movement of the body portion from the decompressed state to the compressed state, the wall is folded along the fold lines; and
an electrode portion connected to the body portion and comprising an electrode for measuring the electrical signal.

2. The sensor of claim 1, wherein the foldable wall comprises an auxetic material.

3. The sensor of claim 1, wherein:
the electrode portion further comprises a foldable wall extending from a first end of the electrode portion to a second end of the electrode portion;
the electrode portion is movable from a unexpanded state to an expanded state by moving the first end of the electrode portion along the longitudinal axis relative to the second end of the electrode portion; and
the wall of the electrode portion comprises fold lines formed therein such that, during movement of the electrode portion from the unexpanded state to the expanded state, the wall of the electrode portion is folded along the fold lines of the electrode portion and thereby causes the electrode portion to expand in a radial direction relative to the longitudinal axis.

4. The sensor of claim 3, wherein the fold lines of the electrode portion are arranged such that, during movement of the electrode portion from the unexpanded state to the expanded state, a distance separating the first end of the electrode portion from the second end of the electrode portion decreases in a direction defined by the longitudinal axis.

5. The sensor of claim 3 wherein the fold lines of the electrode portion define multiple polygonal surface portions of the wall of the electrode portion, the multiple polygonal surface portions comprising a sequence of alternating rectangular and triangular surface portions.

6. The sensor of claim 1, wherein the electrode portion comprises an outer surface and an opposing inner surface facing toward the longitudinal axis, and wherein the electrode is comprised on the inner surface.

7. The sensor of claim 1, wherein the electrode comprises one or more serpentine conductive elements.

8. The sensor of claim 7, wherein the one or more serpentine conductive elements extend in a first direction, and wherein the electrode further comprises one or more serpentine conductive elements extending in a second direction.

9. The sensor of claim 8, wherein the first direction is perpendicular to the second direction.

10. The sensor of claim 1, further comprising one or more electrical conductors connected to the electrode.

11. The sensor of claim 10, wherein the one or more electrical conductors pass through an interior of the body portion.

12. The sensor of claim 10, wherein:
the body portion comprises an outer surface and an opposing inner surface facing toward the longitudinal axis; and
the one or more electrical conductors are in contact with the outer surface of the body portion.

13. The sensor of claim 12, wherein the fold lines of the body portion define at least one polygonal surface portion of the wall of the body portion.

14. The sensor of claim 13, wherein the at least one polygonal surface portion comprises interconnected polygonal surface portions of the wall of the body portion, and wherein the interconnected polygonal surface portions comprise an outer surface of the wall of the body portion.

15. The sensor of claim 13, wherein the at least one polygonal surface portion comprises at least one planar polygonal surface portion.

16.-28. (canceled)

29. The sensor of claim 1, wherein the wall of the body portion is formed by three-dimensional printing.

30. A system for measuring an electrical signal generated by a patient, comprising:
a sensor according to claim 1; and
one or more processors communicative with the sensor and configured to receive electrical conductivity readings from the electrode of the sensor.

31. The system of claim 30, wherein the one or more processors are further configured to:
measure an electrical resistance of a conductive element provided on the sensor; and
based on the measured electrical resistance, determine an amount of rotation of the body portion about the longitudinal axis.

32. The system of claim 30, further comprising a photoplethysmography (PPG) sensor, and wherein the one or more processors are further configured to:
obtain one or more PPG signals from the PPG sensor;
determine one or more electrocardiogram (ECG) signals from the electrical conductivity readings; and
determine a blood pressure based on the one or more PPG signals and the one or more ECG signals.

33.-36. (canceled)

37. A humanoid robot comprising:
a first sensor for generating an ECG reading from a patient;
a second sensor for generating a PPG reading from the patient; and
one or more controllers for determining, based on the ECG reading and the PPG reading, a blood pressure of the patient.
